(19) United States
(12) Patent Application Publication
(10) Pub. No.: US 2019/0224466 A1
(43) Pub. Date: Jul. 25, 2019

(54) Coupling for Medical Devices

(71) Applicant: St. Jude Medical, Cardiology Division, Inc., St. Paul, MN (US)

(72) Inventors: Andrew Oliverius, Eagan, MN (US); John Hong, Prior Lake, MN (US); Xuan Khieu, Maple Grove, MN (US); Taylor Nasby, Lakeville, MN (US); James C. Marrs, Arden Hills, MN (US)

(21) Appl. No.: 16/252,199

(22) Filed: Jan. 18, 2019

Related U.S. Application Data
(60) Provisional application No. 62/619,492, filed on Jan. 19, 2018.

(51) Int. Cl. A61M 39/10; A61B 5/042; A61B 18/14

(52) U.S. Cl. CPC A61M 39/10; A61B 2018/00577; A61B 18/1492; A61B 5/042

(57) ABSTRACT

A coupling for securing a hollow tip to a catheter shaft generally includes a distal portion and a proximal portion. The distal portion includes a first cylindrical body, an outer surface of which includes at least one circumferential groove and at least one flat region. The proximal portion includes a second cylindrical body, an outer surface of which includes at least one channel. A ramp may also be provided at a distal end of the second cylindrical body. A nominal outer diameter of the first cylindrical body is smaller than a nominal outer diameter of the second cylindrical body. Together, the distal portion and the proximal portion define a tunnel extending through and along a length of the coupling.

### CROSS-REFERENCE TO RELATED APPLICATIONS

This application claims the benefit of U.S. provisional application No. 62/619,492, filed 19 Jan. 2018, which is hereby incorporated by reference as though fully set forth herein.

### BACKGROUND

The instant disclosure relates to catheters for use in medical procedures, such as electrophysiology studies. In particular, the instant disclosure relates to a coupling that can be used to join a catheter shaft to a catheter tip, such as a tip electrode in a radiofrequency (RF) ablation catheter.

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart.

A typical electrophysiology catheter includes an elongate shaft and one or more electrodes on the distal end of the shaft. The electrodes may be used for ablation, diagnosis, or the like. Oftentimes, these electrodes include a tip electrode at the distal end of the elongate shaft. One or more thermocouples can be provided at or near the tip electrode for temperature monitoring during an ablation procedure. An exemplary such catheter is the F1exAbility™ ablation catheter of Abbott Laboratories.

Manufacturing such a catheter, however, can be a complex process. For example, verifying proper placement of the thermocouples relative to the tip electrode can be time consuming, slowing the manufacturing process down and making it more expensive.

### BRIEF SUMMARY

Disclosed herein is a catheter including: a catheter shaft defining a lumen; a tip electrode defining a hollow; and a coupling joining the tip electrode to the catheter shaft, wherein a distal portion of the coupling is inserted into the hollow of the tip electrode and a proximal portion of the coupling is inserted into the lumen of the catheter shaft, and wherein the coupling includes a tunnel extending therethrough connecting the lumen of the catheter shaft to the hollow of the tip electrode.

In aspects of the disclosure, the distal portion of the coupling includes a first cylindrical body and the proximal portion of the coupling includes a second cylindrical body, and wherein a nominal outer diameter of the first cylindrical body is smaller than a nominal outer diameter of the second cylindrical body. For instance, the second cylindrical body can include a reduced outer diameter segment and/or at least one circumferential ridge. Further, an outer surface of the second cylindrical body can define at least one channel.

It is also contemplated that the catheter can include: a first thermocouple extending through the tunnel and in thermal contact with tip electrode within the hollow of the tip electrode; and a second thermocouple extending through the at least one channel and in thermal contact with the tip electrode. The at least one channel can include a ramp at a distal portion of the second cylindrical body and/or a well at a distal portion of the second cylindrical body.

In aspects of the disclosure, an outer surface of the first cylindrical body includes at least one circumferential groove. An inner surface of the tip electrode can define at least one circumferential ridge, wherein the at least one ridge is complementary to the at least one circumferential groove.

In additional aspects of the disclosure, an outer surface of the first cylindrical body can include at least one flat region. The at least one flat region can extend along an axial length of the first cylindrical body.

Also disclosed herein is a method of manufacturing a catheter, including the steps of: providing a catheter shaft defining a lumen, a tip electrode defining a hollow, and a coupling including a tunnel extending therethrough; inserting a distal portion of the coupling into the hollow of the tip electrode; inserting a proximal portion of the coupling into the lumen of the catheter shaft; securing the tip electrode to the coupling; and securing the catheter shaft to the coupling. The method can also include inserting a thermocouple through the tunnel and into the hollow of the tip electrode such that the thermocouple is in thermal contact with the tip electrode.

In embodiments of the disclosure, an outer surface of the proximal portion of the coupling includes at least one channel, and the method further includes inserting a thermocouple into the channel such that the thermocouple is in thermal contact with the tip electrode.

It is contemplated that the distal portion of the coupling can include a first cylindrical body and the proximal portion of the coupling can include a second cylindrical body, wherein a nominal outer diameter of the first cylindrical body is smaller than a nominal outer diameter of the second cylindrical body, and wherein inserting a distal portion of the coupling into the hollow of the tip electrode includes inserting the first cylindrical body into the hollow of the tip electrode until the tip electrode abuts the second cylindrical body.

It is also contemplated that an outer surface of the distal portion of the coupling can include at least one circumferential groove, and wherein the at least one circumferential groove defines an extent to which the distal portion of the coupling is inserted into the hollow of the tip electrode. Further, an inner surface of the tip electrode can define at least one circumferential ridge, wherein the at least one ridge is complementary to the at least one circumferential groove.

The instant disclosure also provides a coupling for securing a hollow tip to a catheter shaft, the coupling including: a distal portion including a first cylindrical body, an outer surface of the first cylindrical body including at least one circumferential groove and at least one flat region; and a proximal portion including a second cylindrical body, wherein a nominal outer diameter of the first cylindrical body is smaller than a nominal outer diameter of the second cylindrical body, an outer surface of the second cylindrical body including at least one channel, wherein the distal portion and the proximal portion together define a tunnel extending through and along a length of the coupling. The at least one channel can include a ramp at a distal end of the second cylindrical body.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts a representative electrophysiology catheter.

FIG. 2A is an exploded view of a catheter incorporating a first embodiment of a coupling as disclosed herein.

FIG. 2B is an exploded view of a catheter incorporating a second embodiment of a coupling as disclosed herein.

FIGS. 3A and 4A are perspective views of a first embodiment of a coupling according to aspects of the instant disclosure. More specifically, FIGS. 3A and 4A are two perspective views of a first embodiment of a coupling as disclosed herein as seen from different directions and at different angular rotations.

FIGS. 3B and 4B are perspective views of a second embodiment of a coupling according to aspects of the instant disclosure. More specifically, FIGS. 3B and 4B are two perspective views of a second embodiment of a coupling as disclosed herein as seen from different directions and at different angular rotations.

FIG. 5A depicts the use of a first embodiment of a coupling as disclosed herein in the assembly of a catheter.

FIG. 5B depicts the use of a second embodiment of a coupling as disclosed herein in the assembly of a catheter.

### DETAILED DESCRIPTION

For the sake of illustration, certain embodiments of the disclosure will be explained herein with reference to a radiofrequency (RF) ablation catheter utilized in cardiac ablation procedures. It should be understood, however, that the present teachings may be applied to good advantage in other contexts as well.

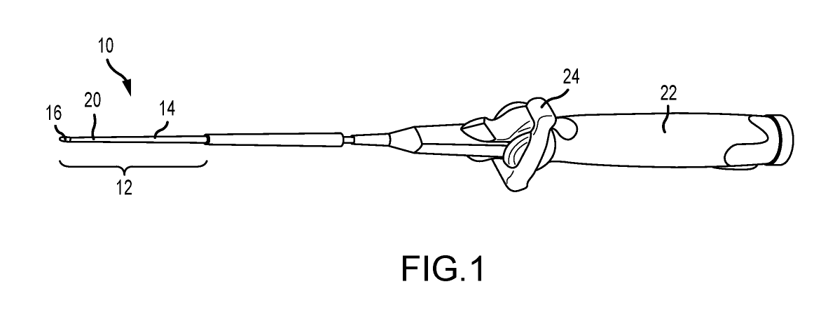

Referring now to the figures, FIG. 1 depicts an embodiment of an electrophysiology (“EP”) catheter 10 according to aspects of the present disclosure. EP catheter 10 includes catheter body 12, which in turn includes a catheter shaft 14 and a tip electrode 16 attached to a distal end of catheter shaft 14.

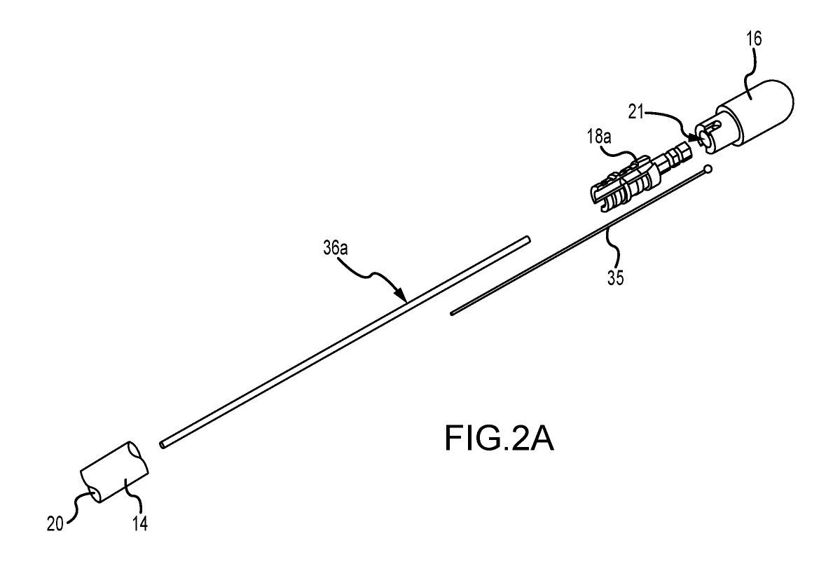

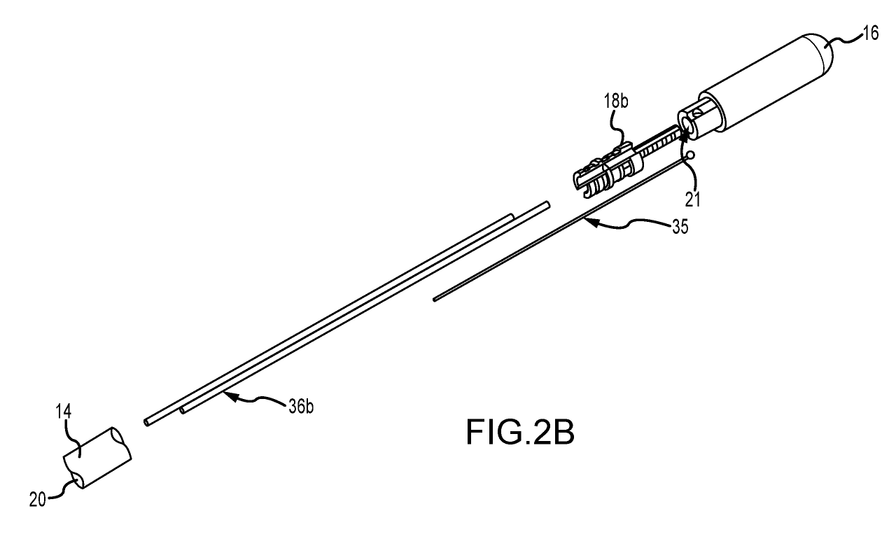

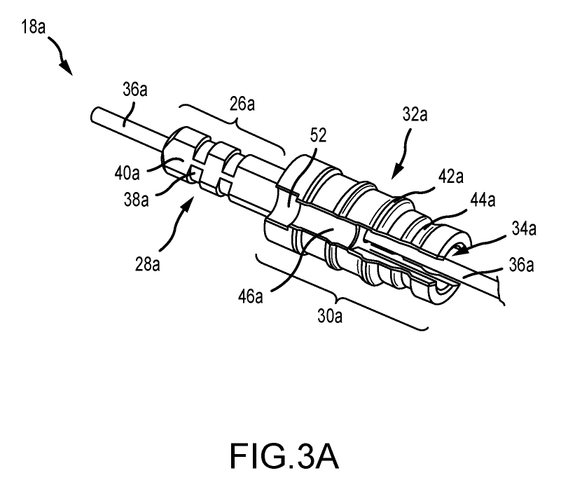

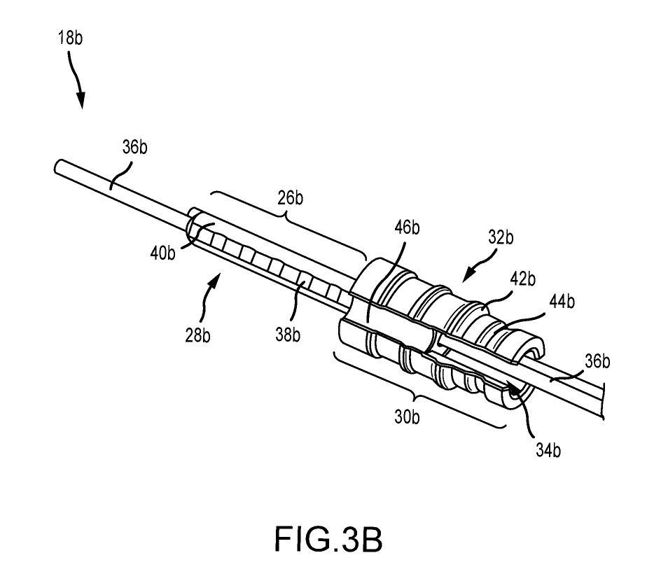

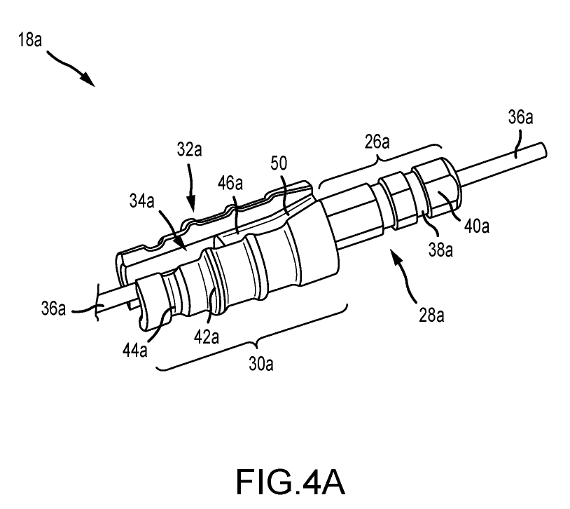

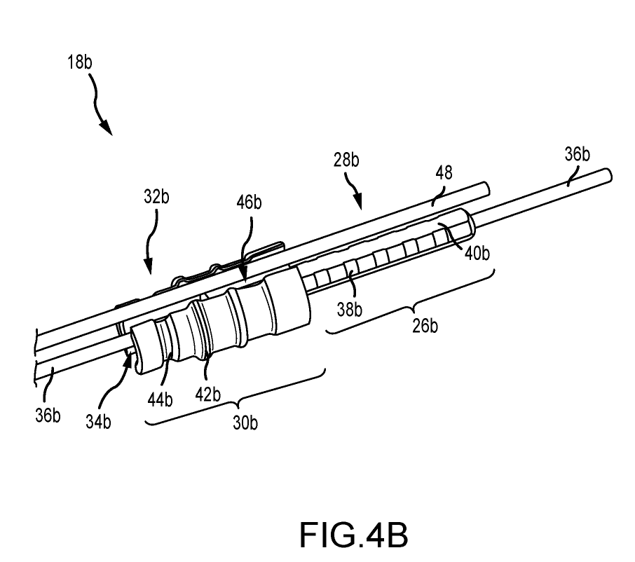

As illustrated in FIGS. 2A and 2B, tip electrode 16 and catheter shaft 14 are joined by a coupling 18 (e.g., 18a, as shown in FIG. 2A, and 18b, as shown in FIG. 2B). Embodiments of coupling 18 are also depicted, for example, in FIGS. 3A, 3B, 4A, and 4B.

In some embodiments, catheter shaft 14 is tubular (e.g., it defines at least one lumen 20 therethrough). It should also be understood that the length of catheter shaft 14 as depicted in FIG. 1 is merely illustrative and may vary without departing from the scope of the instant disclosure. Of course, the overall length of catheter body 12 should be long enough to reach the intended destination within the patient's body (e.g., a location within the patient's heart).

Catheter shaft 14 will typically be made of a biocompatible polymeric material, such as polytetrafluoroethylene (PTFE) tubing (e.g., TEFLON® brand tubing). Of course, other polymeric materials, such as fluorinated ethylene-propylene copolymer (FEP), perfluoroalkoxyethylene (PFA), poly(vinylidene fluoride), poly(ethylene-co-tetrafluoroethylene), and other fluoropolymers, may be utilized. Additional suitable materials for catheter shaft 14 include, without limitation, polyamide-based thermoplastic elastomers (namely poly(ether-block-amide), such as PEBAX®), polyester-based thermoplastic elastomers (e.g., HYTREL®), thermoplastic polyurethanes (e.g., PELLETHANE®, ESTANE®), ionic thermoplastic elastomers, functionalized thermoplastic olefins, and any combinations thereof. In general, suitable materials for catheter shaft 14 may also be selected from various thermoplastics, including, without limitation, polyamides, polyurethanes, polyesters, functionalized polyolefins, polycarbonate, polysulfones, polyimides, polyketones, liquid crystal polymers and any combination thereof. It is also contemplated that the durometer of catheter shaft 14 may vary along its length. In general, the basic construction of catheter shaft 14 will be familiar to those of ordinary skill in the art, and thus will not be discussed in further detail herein except to the extent necessary to understand the instant disclosure.

Tip electrode 16 includes a hollow interior 21 (not visible in FIG. 1, but visible, for example, in FIGS. 2A and 2B). As used herein, the term “hollow interior” means that there is at least one cavity within the interior; the term “hollow core” is used synonymously in this disclosure. Although this cavity can extend throughout the entire length of tip electrode 16 with a substantially constant diameter, the term “hollow” is not intended to be so limited. Thus, the diameter of the cavity can vary along the length of tip electrode 16 and still be considered “hollow” within the meaning of the instant disclosure.

A handle 22 is coupled to catheter body 12. Handle 22 can include a suitable actuator 24 to control the deflection of catheter body 12, for example as described in U.S. Pat. No. 8,369,923, which is hereby incorporated by reference as though fully set forth herein. Various handles and their associated actuators for use in connection with electrophysiology catheters are known, and thus handle 22 will not be described in further detail herein.

FIGS. 3A and 4A depict a first embodiment of coupling 18, designated 19a. FIGS. 3B and 4B depict a second embodiment of coupling 18, designated 19b. The common features of couplings 19a, 19b will be discussed together.

Couplings 19a, 19b generally include a distal portion 26 (e.g., 26a, 26b), which includes a first cylindrical body 28 (e.g., 29a, 29b), and a proximal portion 30 (e.g., 30a, 30b), which includes a second cylindrical body 32 (e.g., 32a, 32b). The nominal outer diameter of first cylindrical body 28 is smaller than the nominal outer diameter of second cylindrical body 32.

For purposes of this disclosure, the phrase “cylindrical body” refers broadly to a body that is generally cylindrical, and is not limited to bodies that are perfectly cylindrical. Thus, a body can include grooves, ridges, flat portions, channels, discontinuities, and the like as discussed herein and still be a “cylindrical body” as that term is used herein. Similarly, the term “nominal outer diameter” refers to the diameter of the cylindrical body as if smooth (e.g., ignoring variations in outer diameter attributable to grooves, ridges, flat portions, channels, discontinuities, and the like as discussed herein).

Coupling 18 further includes a tunnel 34 (e.g., 34a, 34b) extending therethrough. As shown in FIGS. 3A and 3B, and as discussed in greater detail below, tunnels 34a, 34b can be used to pass other components of catheter 10, such as electrode wires 35 (shown in FIGS. 2A and 2B) and/or thermocouples 36 (e.g., 36a, 36b) from the proximal end of coupling 18 to the distal end thereof

First cylindrical body 29a, 29b of coupling 19a, 19b includes at least one circumferential groove 38 (e.g., 39a, 39b). The term “circumferential” is used to herein to mean that the groove 38 extends around at least a portion of the circumference (that is, the perimeter) of the outer surface of first cylindrical body 28. As discussed in greater detail below, circumferential grooves 38 facilitate bonding between coupling 18 and tip electrode 16. Circumferential grooves 38 can also serve as datums for positioning of a thermocouple relative to coupling 18 and/or for positioning coupling 18 relative to tip electrode 16.

First cylindrical body 29a, 29b of coupling 19a, 19b can also include at least one flat region 40 (e.g., 40a, 40b). Flat region 40, which can extend along an axial length of first cylindrical body 28, prevents hydraulic damming of an adhesive during insertion of distal portion 26 of coupling 18 into the hollow interior 21 of tip electrode 16 as described herein.

Second cylindrical body 32a, 32b of coupling 19a, 19b can include at least one circumferential ridge 42 (e.g., 42a, 42b). In analogous fashion to circumferential grooves 38 on first cylindrical body 28, ridges 42 can facilitate bonding between coupling 18 and catheter shaft 14. For example, ridges 42 can increase the bondability between coupling 18 and catheter shaft 14, for example by creating a mechanical lock with the adhesive used to attach coupling 18 to catheter shaft 14.

Second cylindrical body 32a, 32b of coupling 19a, 19b can also include at least one segment 44 (e.g., 44a, 44b) having a reduced outer diameter (e.g., smaller than the nominal outer diameter of second cylindrical body 32). Segment 44 allows for easier access when applying adhesive to bond thermocouple 36 to coupling 18.

In aspects of the disclosure, second cylindrical body 32a, 32b of coupling 19a, 19b also includes a channel 46 (e.g., 46a, 46b) into its outer surface. As discussed in further detail below, channel 46 accommodates an additional thermocouple 48 (shown in FIG. 4B).

The distal end of channel 46 can include a ramp 50 (shown in FIG. 4A), which helps minimize the likelihood that electrode wires 35 (shown in FIGS. 2A and 2B) will be damaged by the relatively sharp edge of tip electrode 16.

In additional aspects of the disclosure, the distal end of channel 46 can include a well 52 (shown in FIG. 3A) to accommodate solder overflow.

Coupling 18 can be micromolded. Micromolding coupling 18 offers certain advantages, such as the creation of well-defined edges that can be used as datums to measure distances, for example when positioning thermocouples 36, 48, thereby increasing the precision and accuracy with which thermocouples 36, 48 can be placed.

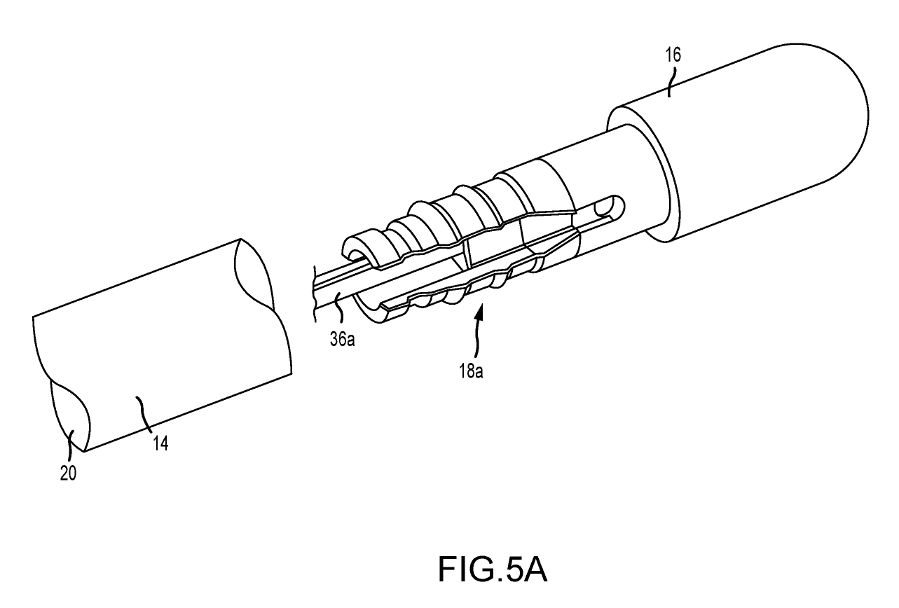

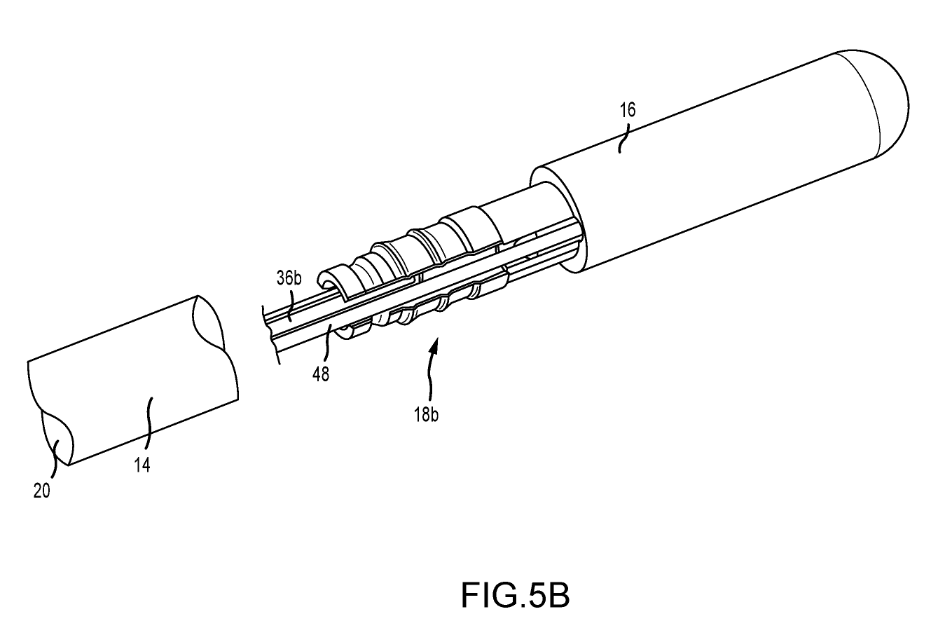

Use of couplings 19a, 19b in the manufacture of catheter 10 will be described with reference to FIGS. 5A and 5B, and can also be understood with reference to FIGS. 2A and 2B. Although various manufacturing steps will be described in a particular order herein, those of ordinary skill in the art will appreciate that the instant teachings are not limited to the order used herein for purposes of explanation. That is, the various manufacturing steps described herein can be rearranged without departing from the scope of the present disclosure.

As seen in FIGS. 5A and 5B, distal portion 26 of coupling 18 is inserted into the hollow interior 21 of tip electrode 16. For example, first cylindrical body 28 is inserted into the hollow interior 21 of tip electrode 16 until tip electrode 16 abuts second cylindrical body 32.

It is also contemplated that an inner surface of tip electrode 16 can define at least one circumferential ridge, complementary to circumferential groove 38 on coupling 18, as a further aid to achieving insertion of coupling 18 into the hollow interior 21 of tip electrode 16 to the desired extent. For example, coupling 18 can be inserted into the hollow interior 21 of tip electrode 16 until the circumferential ridge(s) on the inner surface of tip electrode 16 set into their respective circumferential groove(s) 38 on coupling 18. Alternatively, coupling 18 can be inserted into the hollow interior 21 of tip electrode 16 until circumferential groove(s) 38 on coupling 18 abut their respective circumferential ridge(s) on the inner surface of tip electrode 16.

Thermocouple 36 is inserted through tunnel 34, such that the distal portion of thermocouple 36 extends into the hollow interior 21 of tip electrode 16. More particularly, the distal portion of thermocouple 36 will be in thermal contact with tip electrode 16 within the hollow interior 21 thereof. Similarly, thermocouple 48 can be inserted into channel 46 and into thermal contact with tip electrode 16. Advantageously, the position of thermocouples 36, 48 can be verified using features on coupling 18 (e.g., circumferential grooves 38 on first cylindrical body 28 and/or circumferential ridges 42 on second cylindrical body 32). Once properly positioned, thermocouples 36, 48 can be secured in place using techniques familiar to those of ordinary skill in the art.

Proximal portion 30 of coupling 18 is inserted into catheter shaft 14 (e.g., into lumen 20). For example, second cylindrical body 32 is inserted into lumen 20 of catheter shaft 14 until catheter shaft 14 abuts tip electrode 16. Tunnel 34 thus connects lumen 20 to the hollow interior of tip electrode 16.

Both tip electrode 16 and catheter shaft 14 are secured to coupling 18. For example, a suitable adhesive can be applied to distal portion 26 of coupling 18 prior to insertion thereof into the hollow interior 21 of tip electrode 16; as discussed above, flat region 40 prevents hydraulic damming of the adhesive during the insertion process. A suitable adhesive, such as an ultraviolet-cured adhesive, can also be used to secure catheter shaft 14 to proximal portion 30 of coupling 18.

The use of coupling 18 offers various advantages. For example, it facilitates concentric alignment between tip electrode 16 and catheter shaft 14. As another example, because both tip electrode 16 and catheter shaft 14 are secured to coupling 18, an additional tether wire between tip electrode 16 and catheter shaft 14 can be omitted.

Although several embodiments of this invention have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A catheter comprising:
a catheter shaft defining a lumen;
a tip electrode defining a hollow; and
a coupling joining the tip electrode to the catheter shaft, wherein a distal portion of the coupling is inserted into the hollow of the tip electrode and a proximal portion of the coupling is inserted into the lumen of the catheter shaft, and
wherein the coupling includes a tunnel extending therethrough connecting the lumen of the catheter shaft to the hollow of the tip electrode.

2. The catheter according to claim 1, wherein the distal portion of the coupling comprises a first cylindrical body and the proximal portion of the coupling comprises a second cylindrical body, and wherein a nominal outer diameter of the first cylindrical body is smaller than a nominal outer diameter of the second cylindrical body.

3. The catheter according to claim 2, wherein the second cylindrical body comprises a reduced outer diameter segment.

4. The catheter according to claim 2, wherein the second cylindrical body comprises at least one circumferential ridge.

5. The catheter according to claim 2, wherein an outer surface of the second cylindrical body defines at least one channel.

6. The catheter according to claim 1, further comprising:
a first thermocouple extending through the tunnel and in thermal contact with tip electrode within the hollow of the tip electrode; and
a second thermocouple extending through the at least one channel and in thermal contact with the tip electrode.

7. The catheter according to claim 5, wherein the at least one channel comprises a ramp at a distal portion of the second cylindrical body.

8. The catheter according to claim 5, wherein the at least one channel comprises a well at a distal portion of the second cylindrical body.

9. The catheter according to claim 2, wherein an outer surface of the first cylindrical body comprises at least one circumferential groove.

10. The catheter according to claim 9, wherein an inner surface of the tip electrode defines at least one circumferential ridge, wherein the at least one ridge is complementary to the at least one circumferential groove.

11. The catheter according to claim 2, wherein an outer surface of the first cylindrical body comprises at least one flat region.

12. The catheter according to claim 11, wherein the at least one flat region extends along an axial length of the first cylindrical body.

13. A method of manufacturing a catheter, comprising:
providing a catheter shaft defining a lumen, a tip electrode defining a hollow, and a coupling including a tunnel extending therethrough;
inserting a distal portion of the coupling into the hollow of the tip electrode;
inserting a proximal portion of the coupling into the lumen of the catheter shaft;
securing the tip electrode to the coupling; and
securing the catheter shaft to the coupling.

14. The method according to claim 13, further comprising inserting a thermocouple through the tunnel and into the hollow of the tip electrode such that the thermocouple is in thermal contact with the tip electrode.

15. The method according to claim 13, wherein an outer surface of the proximal portion of the coupling comprises at least one channel, the method further comprising inserting a thermocouple into the channel such that the thermocouple is in thermal contact with the tip electrode.

16. The method according to claim 13, wherein the distal portion of the coupling comprises a first cylindrical body and the proximal portion of the coupling comprises a second cylindrical body, wherein a nominal outer diameter of the first cylindrical body is smaller than a nominal outer diameter of the second cylindrical body, and wherein inserting a distal portion of the coupling into the hollow of the tip electrode comprises inserting the first cylindrical body into the hollow of the tip electrode until the tip electrode abuts the second cylindrical body.

17. The method according to claim 13, wherein an outer surface of the distal portion of the coupling comprises at least one circumferential groove, and wherein the at least one circumferential groove defines an extent to which the distal portion of the coupling is inserted into the hollow of the tip electrode.

18. The method according to claim 17, wherein an inner surface of the tip electrode defines at least one circumferential ridge, wherein the at least one ridge is complementary to the at least one circumferential groove.

19. A coupling for securing a hollow tip to a catheter shaft, the coupling comprising:
a distal portion comprising a first cylindrical body, an outer surface of the first cylindrical body including at least one circumferential groove and at least one flat region; and
a proximal portion comprising a second cylindrical body, wherein a nominal outer diameter of the first cylindrical body is smaller than a nominal outer diameter of the second cylindrical body, an outer surface of the second cylindrical body including at least one channel,
wherein the distal portion and the proximal portion together define a tunnel extending through and along a length of the coupling.

20. The coupling according to claim 20, wherein the at least one channel comprises a ramp at a distal end of the second cylindrical body.
